SEQUENCE LISTING (12 sequences)

SEQ ID NO: 1 - DNA, 3845 bp, Homo sapiens
  CDS (37)..(3453)
  misc_feature: Human Tie-1
cgctcgtcctggctggcctgggtcggcctctggagtatggtctggcgggtgccccctttcttgctccccatcctcttcttggcttctcatgtgggcgcggcggtggacctgacgctgctggccaacctgcggctcacggacccccagcgcttcttcctgacttgcgtgtctggggaggccggggcggggaggggctcggacgcctggggcccgcccctgctgctggagaaggacgaccgtatcgtgcgcaccccgcccgggccacccctgcgcctggcgcgcaacggttcgcaccaggtcacgcttcgcggcttctccaagccctcggacctcgtgggcgtcttctcctgcgtgggcggtgctggggcgcggcgcacgcgcgtcatctacgtgcacaacagccctggagcccacctgcttccagacaaggtcacacacactgtgaacaaaggtgacaccgctgtactttctgcacgtgtgcacaaggagaagcagacagacgtgatctggaagagcaacggatcctacttctacaccctggactggcatgaagcccaggatgggcggttcctgctgcagctcccaaatgtgcagccaccatcgagcggcatctacagtgccacttacctggaagccagccccctgggcagcgccttctttcggctcatcgtgcggggttgtggggctgggcgctgggggccaggctgtaccaaggagtgcccaggttgcctacatggaggtgtctgccacgaccatgacggcgaatgtgtatgcccccctggcttcactggcacccgctgtgaacaggcctgcagagagggccgttttgggcagagctgccaggagcagtgcccaggcatatcaggctgccggggcctcaccttctgcctcccagacccctatggctgctcttgtggatctggctggagaggaagccagtgccaagaagcttgtgcccctggtcattttggggctgattgccgactccagtgccagtgtcagaatggtggcacttgtgaccggttcagtggttgtgtctgcccctctgggtggcatggagtgcactgtgagaagtcagaccggatcccccagatcctcaacatggcctcagaactggagttcaacttagagacgatgccccggatcaactgtgcagctgcagggaaccccttccccgtgcggggcagcatagagctacgcaagccagacggcactgtgctcctgtccaccaaggccattgtggagccagagaagaccacagctgagttcgaggtgccccgcttggttcttgcggacagtgggttctgggagtgccgtgtgtccacatctggcggccaagacagccggcgcttcaaggtcaatgtgaaagtgccccccgtgcccctggctgcacctcggctcctgaccaagcagagccgccagcttgtggtctccccgctggtctcgttctctggggatggacccatctccactgtccgcctgcactaccggccccaggacagtaccatggactggtcgaccattgtggtggaccccagtgagaacgtgacgttaatgaacctgaggccaaagacaggatacagtgttcgtgtgcagctgagccggccaggggaaggaggagagggggcctgggggcctcccaccctcatgaccacagactgtcctgagcctttgttgcagccgtggttggagggctggcatgtggaaggcactgaccggctgcgagtgagctggtccttgcccttggtgcccgggccactggtgggcgacggtttcctgctgcgcctgtgggacgggacacgggggcaggagcggcgggagaacgtctcatccccccaggcccgcactgccctcctgacgggactcacgcctggcacccactaccagctggatgtgcagctctaccactgcaccctcctgggcccggcctcgccccctgcacacgtgcttctgccccccagtgggcctccagccccccgacacctccacgcccaggccctctcagactccgagatccagctgacatggaagcacccggaggctctgcctgggccaatatccaagtacgttgtggaggtgcaggtggctgggggtgcaggagacccactgtggatagacgtggacaggcctgaggagacaagcaccatcatccgtggcctcaacgccagcacgcgctacctcttccgcatgcgggccagcattcaggggctcggggactggagcaacacagtagaagagtccaccctgggcaacgggctgcaggctgagggcccagtccaagagagccgggcagctgaagagggcctggatcagcagctgatcctggcggtggtgggctccgtgtctgccacctgcctcaccatcctggccgcccttttaaccctggtgtgcatccgcagaagctgcctgcatcggagacgcaccttcacctaccagtcaggctcgggcgaggagaccatcctgcagttcagctcagggaccttgacacttacccggcggccaaaactgcagcccgagcccctgagctacccagtgctagagtgggaggacatcacctttgaggacctcatcggggaggggaacttcggccaggtcatccgggccatgatcaagaaggacgggctgaagatgaacgcagccatcaaaatgctgaaagagtatgcctctgaaaatgaccatcgtgactttgcgggagaactggaagttctgtgcaaattggggcatcaccccaacatcatcaacctcctgggggcctgtaagaaccgaggttacttgtatatcgctattgaatatgccccctacgggaacctgctagattttctgcggaaaagccgggtcctagagactgacccagcttttgctcgagagcatgggacagcctctacccttagctcccggcagctgctgcgtttcgccagtgatgcggccaatggcatgcagtacctgagtgagaagcagttcatccacagggacctggctgcccggaatgtgctggtcggagagaacctagcctccaagattgcagacttcggcctttctcggggagaggaggtttatgtgaagaagacgatggggcgtctccctgtgcgctggatggccattgagtccctgaactacagtgtctataccaccaagagtgatgtctggtcctttggagtccttctttgggagatagtgagccttggaggtacaccctactgtggcatgacctgtgccgagctctatgaaaagctgccccagggctaccgcatggagcagcctcgaaactgtgacgatgaagtgtacgagctgatgcgtcagtgctggcgggaccgtccctatgagcgacccccctttgcccagattgcgctacagctaggccgcatgctggaagccaggaaggcctatgtgaacatgtcgctgtttgagaacttcacttacgcgggcattgatgccacagctgaggaggcctgagctgccatccagccagaacgtggctctgctggccggagcaaactctgctgtctaacctgtgaccagtctgacccttacagcctctgacttaagctgcctcaaggaatttttttaacttaagggagaaaaaaagggatctggggatggggtgggcttaggggaactgggttcccatgctttgtaggtgtctcatagctatcctgggcatccttctttctagttcagctgccccacaggtgtgtttcccatcccactgctcccccaacacaaacccccactccagctccttcgcttaagccagcactcacaccactaacatgccctgttcagctactcccactcccggcctgtcattcagaaaaaaataaatgttctaataagctccaaaaaaa

SEQ ID NO: 2 - protein, 1138 aa, Homo sapiens
  misc_feature: Human Tie-1
MVWRVPPFLLPILFLASHVGAAVDLTLLANLRLTDPQRFFLTCVSGEAGAGRGSDAWGPPLLLEKDDRIVRTPPGPPLRLARNGSHQVTLRGFSKPSDLVGVFSCVGGAGARRTRVIYVHNSPGAHLLPDKVTHTVNKGDTAVLSARVHKEKQTDVIWKSNGSYFYTLDWHEAQDGRFLLQLPNVQPPSSGIYSATYLEASPLGSAFFRLIVRGCGAGRWGPGCTKECPGCLHGGVCHDHDGECVCPPGFTGTRCEQACREGRFGQSCQEQCPGISGCRGLTFCLPDPYGCSCGSGWRGSQCQEACAPGHFGADCRLQCQCQNGGTCDRFSGCVCPSGWHGVHCEKSDRIPQILNMASELEFNLETMPRINCAAAGNPFPVRGSIELRKPDGTVLLSTKAIVEPEKTTAEFEVPRLVLADSGFWECRVSTSGGQDSRRFKVNVKVPPVPLAAPRLLTKQSRQLVVSPLVSFSGDGPISTVRLHYRPQDSTMDWSTIVVDPSENVTLMNLRPKTGYSVRVQLSRPGEGGEGAWGPPTLMTTDCPEPLLQPWLEGWHVEGTDRLRVSWSLPLVPGPLVGDGFLLRLWDGTRGQERRENVSSPQARTALLTGLTPGTHYQLDVQLYHCTLLGPASPPAHVLLPPSGPPAPRHLHAQALSDSEIQLTWKHPEALPGPISKYVVEVQVAGGAGDPLWIDVDRPEETSTIIRGLNASTRYLFRMRASIQGLGDWSNTVEESTLGNGLQAEGPVQESRAAEEGLDQQLILAVVGSVSATCLTILAALLTLVCIRRSCLHRRRTFTYQSGSGEETILQFSSGTLTLTRRPKLQPEPLSYPVLEWEDITFEDLIGEGNFGQVIRAMIKKDGLKMNAAIKMLKEYASENDHRDFAGELEVLCKLGHHPNIINLLGACKNRGYLYIAIEYAPYGNLLDFLRKSRVLETDPAFAREHGTASTLSSRQLLRFASDAANGMQYLSEKQFIHRDLAARNVLVGENLASKIADFGLSRGEEVYVKKTMGRLPVRWMAIESLNYSVYTTKSDVWSFGVLLWEIVSLGGTPYCGMTCAELYEKLPQGYRMEQPRNCDDEVYELMRQCWRDRPYERPPFAQIALQLGRMLEARKAYVNMSLFENFTYAGIDATAEEA

SEQ ID NO: 3 - DNA, 4138 bp, Homo sapiens
  CDS (149)..(3523)
  misc_feature: Human Tie-2
cttctgtgctgttccttcttgcctctaacttgtaaacaagacgtactaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggaactgtggaaggtgccatggacttgatcttgatcaattccctacctcttgtatctgatgctgaaacatctctcacctgcattgcctctgggtggcgcccccatgagcccatcaccataggaagggactttgaagccttaatgaaccagcaccaggatccgctggaagttactcaagatgtgaccagagaatgggctaaaaaagttgtttggaagagagaaaaggctagtaagatcaatggtgcttatttctgtgaagggcgagttcgaggagaggcaatcaggatacgaaccatgaagatgcgtcaacaagcttccttcctaccagctactttaactatgactgtggacaagggagataacgtgaacatatctttcaaaaaggtattgattaaagaagaagatgcagtgatttacaaaaatggttccttcatccattcagtgccccggcatgaagtacctgatattctagaagtacacctgcctcatgctcagccccaggatgctggagtgtactcggccaggtatataggaggaaacctcttcacctcggccttcaccaggctgatagtccggagatgtgaagcccagaagtggggacctgaatgcaaccatctctgtactgcttgtatgaacaatggtgtctgccatgaagatactggagaatgcatttgccctcctgggtttatgggaaggacgtgtgagaaggcttgtgaactgcacacgtttggcagaacttgtaaagaaaggtgcagtggacaagagggatgcaagtcttatgtgttctgtctccctgacccctatgggtgttcctgtgccacaggctggaagggtctgcagtgcaatgaagcatgccaccctggtttttacgggccagattgtaagcttaggtgcagctgcaacaatggggagatgtgtgatcgcttccaaggatgtctctgctctccaggatggcaggggctccagtgtgagagagaaggcataccgaggatgaccccaaagatagtggatttgccagatcatatagaagtaaacagtggtaaatttaatcccatttgcaaagcttctggctggccgctacctactaatgaagaaatgaccctggtgaagccggatgggacagtgctccatccaaaagactttaaccatacggatcatttctcagtagccatattcaccatccaccggatcctcccccctgactcaggagtttgggtctgcagtgtgaacacagtggctgggatggtggaaaagcccttcaacatttctgttaaagttcttccaaagcccctgaatgccccaaacgtgattgacactggacataactttgctgtcatcaacatcagctctgagccttactttggggatggaccaatcaaatccaagaagcttctatacaaacccgttaatcactatgaggcttggcaacatattcaagtgacaaatgagattgttacactcaactatttggaacctcggacagaatatgaactctgtgtgcaactggtccgtcgtggagagggtggggaagggcatcctggacctgtgagacgcttcacaacagcttctatcggactccctcctccaagaggtctaaatctcctgcctaaaagtcagaccactctaaatttgacctggcaaccaatatttccaagctcggaagatgacttttatgttgaagtggagagaaggtctgtgcaaaaaagtgatcagcagaatattaaagttccaggcaacttgacttcggtgctacttaacaacttacatcccagggagcagtacgtggtccgagctagagtcaacaccaaggcccagggggaatggagtgaagatctcactgcttggacccttagtgacattcttcctcctcaaccagaaaacatcaagatttccaacattacacactcctcggctgtgatttcttggacaatattggatggctattctatttcttctattactatccgttacaaggttcaaggcaagaatgaagaccagcacgttgatgtgaagataaagaatgccaccatcattcagtatcagctcaagggcctagagcctgaaacagcataccaggtggacatttttgcagagaacaacatagggtcaagcaacccagccttttctcatgaactggtgaccctcccagaatctcaagcaccagcggacctcggaggggggaagatgctgcttatagccatccttggctctgctggaatgacctgcctgactgtgctgttggcctttctgatcatattgcaattgaagagggcaaatgtgcaaaggagaatggcccaagccttccaaaacgtgagggaagaaccagctgtgcagttcaactcagggactctggccctaaacaggaaggtcaaaaacaacccagatcctacaatttatccagtgcttgactggaatgacatcaaatttcaagatgtgattggggagggcaattttggccaagttcttaaggcgcgcatcaagaaggatgggttacggatggatgctgccatcaaaagaatgaaagaatatgcctccaaagatgatcacagggactttgcaggagaactggaagttctttgtaaacttggacaccatccaaacatcatcaatctcttaggagcatgtgaacatcgaggctacttgtacctggccattgagtacgcgccccatggaaaccttctggacttccttcgcaagagccgtgtgctggagacggacccagcatttgccattgccaatagcaccgcgtccacactgtcctcccagcagctccttcacttcgctgccgacgtggcccggggcatggactacttgagccaaaaacagtttatccacagggatctggctgccagaaacattttagttggtgaaaactatgtggcaaaaatagcagattttggattgtcccgaggtcaagaggtgtacgtgaaaaagacaatgggaaggctcccagtgcgctggatggccatcgagtcactgaattacagtgtgtacacaaccaacagtgatgtatggtcctatggtgtgttactatgggagattgttagcttaggaggcacaccctactgcgggatgacttgtgcagaactctacgagaagctgccccagggctacagactggagaagcccctgaactgtgatgatgaggtgtatgatctaatgagacaatgctggcgggagaagccttatgagaggccatcatttgcccagatattggtgtccttaaacagaatgttagaggagcgaaagacctacgtgaataccacgctttatgagaagtttacttatgcaggaattgactgttctgctgaagaagcggcctaggacagaacatctgtataccctctgtttccctttcactggcatgggagacccttgacaactgctgagaaaacatgcctctgccaaaggatgtgatatataagtgtacatatgtgctggaattctaacaagtcataggttaatatttaagacactgaaaaatctaagtgatataaatcagattcttctctctcattttatccctcacctgtagcatgccagtcccgtttcatttagtcatgtgaccactctgtcttgtgtttccacagcctgcaagttcagtccaggatgctaacatctaaaaatagacttaaatctcattgcttacaagcctaagaatctttagagaagtatacataagtttaggataaaataatgggattttcttttcttttctctggtaatattgacttgtatattttaagaaataacagaaagcctgggtgacatttgggagacatgtgacatttatatattgaattaatatccctacatgtattgcacattgtaaaaagttttagttttgatgagttgtgagtttaccttgtatactgtaggcacactttgcactgatatatcatgagtgaataaatgtcttgcctactcaaaaaaaaaaaa

SEQ ID NO: 4 - protein, 1124 aa, Homo sapiens
  misc_feature: Human Tie-2
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIPRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATIIQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 5 - DNA, 3041 bp, Homo sapiens
  misc_feature: Human angiopoietin 1 (ANG-1), mRNA
  misc_feature (96)..(665): FBG; Region Fibrinogen-related domains (FReDs)
  CDS (96)..(674)
gaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaatggaatgttctatactgcgggacaaaaccatggaaaactgaatgggataaagtggcactacttcaaagggcccagttactccttacgttccacaactatgatgattcgacctttagatttttgaaagcgcaatgtcagaagcgattatgaaagcaacaaagaaatccggagaagctgccaggtgagaaactgtttgaaaacttcagaagcaaacaatattgtctcccttccagcaataagtggtagttatgtgaagtcaccaaggttcttgaccgtgaatctggagccgtttgagttcacaagagtctctacttggggtgacagtgctcacgtggctcgactatagaaaactccactgactgtcgggctttaaaaagggaagaaactgctgagcttgctgtgcttcaaactactactggaccttattttggaactatggtagccagatgataaatatggttaatttcatgtaaaacagaaaaaaagagtgaaaaagagaatatacatgaagaatagaaacaagcctgccataatcctttggaaaagatgtattataccagtgaaaaggcgttatatctatgcaaacctactaacaaattatactgttgcacaattttgataaaaatttagaacagcattgtcctctgagttggttaaatgttaatggatttcagaagcctaattccagtatcatacttactagttgatttctgcttacccatcttcaaatgaaaattccatttttgtaagccataatgaactgtagtacatggacaataagtgtgtggtagaaacaaactccattactctgatttttgatacagttttcagaaaaagaaatgaacataatcaagtaaggatgtatgtggtgaaaacttaccacccccatactatggttttcatttactctaaaaactgattgaatgatatataaatatatttatagcctgagtaaagttaaaagaatgtaaaatatatcatcaagttcttaaaataatatacatgcatttaatatttcctttgatattatacaggaaagcaatattttggagtatgttaagttgaagtaaaaccaagtactctggagcagttcattttacagtatctacttgcatgtgtatacatacatgtaacttcattattttaaaaatatttttagaactccaatactcaccctgttatgtcttgctaatttaaattttgctaattaactgaaacatgcttaccagattcacactgttccagtgtctataaaagaaacactttgaagtctataaaaaataaaataattataaatatcattgtacatagcatgtttatatctgcaaaaaacctaatagctaattaatctggaatatgcaacattgtccttaattgatgcaaataacacaaatgctcaaagaaatctactatatcccttaatgaaatacatcattcttcatatatttctccttcagtccattcccttaggcaatttttaatttttaaaaattattatcaggggagaaaaattggcaaaactattatatgtaagggatatatatatacaaaaagaaaattaatcatagtcacctgactaagaaattctgactgctagttgccataaataactcaatggaaatattcctatgggataatgtattttaagtgaatttttggggtgcttgaagttactgcattattttatcaagaagtcttctctgcctgtaagtgtccaaggttatgacagtaaacagtttttattaaaacatgagtcactatgggatgagaaaattgaaataaagctactgggcctcctctcataaaagagacagttgttggcaaggtagcaataccagtttcaaacttggtgacttgatccactatgccttaatggtttcctccatttgagaaaataaagctattcacattgttaagaaaaatactttttaaagtttaccatcaagtcttttttatatttatgtgtctgtattctacccctttttgccttacaagtgatatttgcaggtattataccatttttctattcttggtggcttcttcatagcaggtaagcctctccttctaaaaacttctcaactgttttcatttaagggaaagaaaatgagtattttgtccttttgtgttcctacagacactttcttaaaccagtttttggataaagaatactatttccaaactcatattacaaaaacaaaataaaataataaaaaaagaaagcatgatatttactgttttgttgtctgggtttgagaaatgaaatattgtttccaattatttataataaatcagtataaaatgttttatgattgttatgtgtattatgtaatacgtacatgtttatggcaatttaacatgtgtattcttttcatttaattgtttcagaataggataattaggtattcgaattttgtctttaaaattcatgtggtttctatgcaaagttcttcatatcatcacaacattatttgatttaaataaaattgaaagt

SEQ ID NO: 6 - protein, 192 aa, Homo sapiens
  misc_feature: Human angiopoietin 1 (ANG-1), mRNA
  misc_feature (96)..(665): FBG; Region Fibrinogen-related domains (FReDs)
MPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 7 - DNA, 2269 bp, Homo sapiens
  misc_feature: Human angiopoietin 2 (ANG-2), mRNA
  CDS (350)..(1840)
tgggttggtgtttatctcctcccagccttgagggagggaacaacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgtttcagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagctgagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcaaaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacaaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcct

SEQ ID NO: 8 - protein, 496 aa, Homo sapiens
  misc_feature: Human angiopoietin 2 (ANG-2), mRNA
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 9 - DNA, 1957 bp, Homo sapiens
  misc_feature: Human angiopoietin-3 (ANG-3), mRNA
  misc_feature (1497)..(1497): n= a or g or t or c
  CDS (106)..(1617)
ggtgcagctgcaggcaagcctggccactgttggctgcagcaggacatcccaggcacagcccctagggctctgagcagacatccctcgccattgacacatcttcagatgctctcccaactagccatgctgcagggcagcctcctccttgtggttgccaccatgtctgtggctcaacagacaaggcaggaggcggataggggctgcgagacacttgtagtccagcacggccactgtagctacaccttcttgctgcccaagtctgagccctgccctccggggcctgaggtctccagggactccaacaccctccagagagaatcactggccaacccactgcacctggggaagttgcccacccagcaggtgaaacagctggagcaggcactgcagaacaacacgcagtggctgaagaagctagagagggccatcaagacgatcttgaggtcgaagctggagcaggtccagcagcaaatggcccagaatcagacggcccccatgctagagctgggcaccagcctcctgaaccagaccactgcccagatccgcaagctgaccgacatggaggctcagctcctgaaccagacatcaagaatggatgcccagatgccagagacctttctgtccaccaacaagctggagaaccagctgctgctacagaggcagaagctccagcagcttcagggccaaaacagcgcgctcgagaagcggttgcaggccctggagaccaagcagcaggaggagctggccagcatcctcagcaagaaggcgaagctgctgaacacgctgagccgccagagcgccgccctcaccaacatcgagcgcggcctgcgcggtgtcaggcacaactccagcctcctgcaggaccagcagcacagcctgcgccagctgctggtgttgttgcggcacctggtgcaagaaagggctaacgcctcggccccggccttcataatggcaggtgagcaggtgttccaggactgtgcagagatccagcgctctggggccagtgccagtggtgtgtacaccatccaggtgtccaatgcaacgaagcccaggaaggtgttctgtgacctgcagagcagtggaggcaggtggaccctcatccagcgccgtgagaatggcaccgtgaattttcagcggaactggaaggattacaaacagggcttcggagacccagctggggagcactggctgggcaatgaagtggtgcaccagctcaccagaagggcagcctactctctgcgtgtggagctgcaagactgggaaggccacgaggcctatgcccagtacgaacatttccacctgggcagtgagaaccagctatacaggctttctgtggtcgggtacagcggctcagcagggcgccagagcagcctggtcctgcagaacaccagctttagcacccttgactcagacaacgaccactgtctctgcaagtgtgcccaagtgatgtctggagggtggtggtttgacgcctgtggcctgtcaaacctcaacggngtctactaccacgctcccgacaacaagtacaagatggacggcatccgctggcactacttcaagggccccagctactcactgcgtgcctctcgcatgatgatacggcctttggacatctaacgagcagctgtgccagaggctggaccacacaggagaagctcggacttggcactcctggacaacctggacccagatgcaagacactgtgccaccgccttccctgacaccctgggcttcctgagccagccctccttgacccagaagtccagaagggtcatctgcccccccactcccctccgtctgtgacatggagggtgttcggggcccatccctctgatgtagtcctcgcccctcttctctccctcccccttcaggggctccctgcctgagggtcacagtaccttgaatgggctgagaacagaccaaaaaaaaaaaaaaaaaaaaaaaaaaaaaaaaaaaa

SEQ ID NO: 10 - protein, 503 aa, Homo sapiens
  misc_feature: Human angiopoietin-3 (ANG-3), mRNA
  misc_feature (1497)..(1497): n= a or g or t or c
MLSQLAMLQGSLLLVVATMSVAQQTRQEADRGCETLVVQHGHCSYTFLLPKSEPCPPGPEVSRDSNTLQRESLANPLHLGKLPTQQVKQLEQALQNNTQWLKKLERAIKTILRSKLEQVQQQMAQNQTAPMLELGTSLLNQTTAQIRKLTDMEAQLLNQTSRMDAQMPETFLSTNKLENQLLLQRQKLQQLQGQNSALEKRLQALETKQQEELASILSKKAKLLNTLSRQSAALTNIERGLRGVRHNSSLLQDQQHSLRQLLVLLRHLVQERANASAPAFIMAGEQVFQDCAEIQRSGASASGVYTIQVSNATKPRKVFCDLQSSGGRWTLIQRRENGTVNFQRNWKDYKQGFGDPAGEHWLGNEVVHQLTRRAAYSLRVELQDWEGHEAYAQYEHFHLGSENQLYRLSVVGYSGSAGRQSSLVLQNTSFSTLDSDNDHCLCKCAQVMSGGWWFDACGLSNLNGVYYHAPDNKYKMDGIRWHYFKGPSYSLRASRMMIRPLDI

SEQ ID NO: 11 - DNA, 1512 bp, Homo sapiens
  misc_feature: Human angiopoietin 4 (ANG-4), mRNA
  CDS (2)..(1510)
catgctctcccagctagccatgctgcagggcagcctcctccttgtggttgccaccatgtctgtggctcaacagacaaggcaggaggcggataggggctgcgagacacttgtagtccagcacggccactgtagctacaccttcttgctgcccaagtctgagccctgccctccggggcctgaggtctccagggactccaacaccctccagagagaatcactggccaacccactgcacctggggaagttgcccacccagcaggtgaaacagctggagcaggcactgcagaacaacacgcagtggctgaagaagctagagagggccatcaagacgatcttgaggtcgaagctggagcaggtccagcagcaaatggcccagaatcagacggcccccatgctagagctgggcaccagcctcctgaaccagaccactgcccagatccgcaagctgaccgacatggaggctcagctcctgaaccagacatcaagaatggatgcccagatgccagagacctttctgtccaccaacaagctggagaaccagctgctgctacagaggcagaagctccagcagcttcagggccaaaacagcgcgctcgagaagcggttgcaggccctggagaccaagcagcaggaggagctggccagcatcctcagcaagaaggcgaagctgctgaacacgctgagccgccagagcgccgccctcaccaacatcgagcgcggcctgcgcggtgtcaggcacaactccagcctcctgcaggaccagcagcacagcctgcgccagctgctggtgttgttgcggcacctggtgcaagaaagggctaacgcctcggccccggccttcataatggcaggtgagcaggtgttccaggactgtgcagagatccagcgctctggggccagtgccagtggtgtctacaccatccaggtgtccaatgcaacgaagcccaggaaggtgttctgtgacctgcagagcagtggaggcaggtggaccctcatccagcgccgtgagaatggcaccgtgaattttcagcggaactggaaggattacaaacagggcttcggagacccagctggggagcactggctgggcaatgaagtggtgcaccagctcaccagaagggcagcctactctctgcgtgtggagctgcaagactgggaaggccacgaggcctatgcccagtacgaacatttccacctgggcagtgagaaccagctatacaggctttctgtggtcgggtacagcggctcagcagggcgccagagcagcctggtcctgcagaacaccagctttagcacccttgactcagacaacgaccactgtctctgcaagtgtgcccaggtgatgtctggagggtggtggtttgacgcctgtggcctgtcaaacctcaacggcgtctactaccacgctcccgacaacaagtacaagatggacggcatccgctggcactacttcaagggccccagctactcactgcgtgcctctcgcatgatgatacggcctttggacatcta

SEQ ID NO: 12 - protein, 503 aa, Homo sapiens; identical to SEQ ID NO: 10
  misc_feature: Human angiopoietin 4 (ANG-4), mRNA

(19) United States
(12) Patent Application Publication
(10) Pub. No.: US 2007/0280947 A1
(43) Pub. Date: Dec. 6, 2007

(54) Tie Receptor and Tie Ligand Materials and Methods for Modulating Female Fertility

(75) Inventors: Kari Alitalo, Helsinki (FI); Pirjo Laakkonen, Helsinki (FI); Hajime Kubo, Kyoto (JP); Kirsi Sainio, Helsinki (FI)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LICENTIA, LTD., Helsinki (FI)

(21) Appl. No.: 11/630,531

(22) PCT Filed: Jun. 27, 2005
(86) PCT No.: PCT/EP05/06906

§ 371 Date: Aug. 15, 2007

Related U.S. Application Data
(60) Provisional application No. 60/582,858, filed on Jun. 25, 2004.

(51) Int. Cl. A61K 39/395; A61K 38/00; A61K 48/00; A61P 15/00; G01N 33/567; G01N 33/573

(52) U.S. Cl. 424/146.1; 424/130.1; 424/178.1; 435/7.21; 435/7.4; 514/12; 514/44

(57) ABSTRACT

The present invention provides materials and methods involving Tie receptors and Angiopoietin ligands for modulating female fertility in mammals, including humans. Materials and methods for inhibiting fertility (e.g., for contraception) or for enhancing fertility (e.g., treating infertility) are contemplated.

### CROSS REFERENCE TO RELATED APPLICATIONS

The present application claims the priority benefit of U.S. Provisional Application No. 60/582,858, filed Jun. 25, 2004, incorporated herein by reference in its entirety.

### FIELD OF THE INVENTION

The present invention provides materials and methods for modulating (inhibiting or enhancing) female fertility in mammals, including humans.

### BACKGROUND OF THE INVENTION

Angiogenesis is the process in which new blood vessels are formed by capillary sprouting from the established vascular network in response to angiogenic stimuli. Following the proliferation and migration of endothelial cells, vessels need to be stabilized and matured into fully functional vessels in a process that requires recruitment and interaction of endothelial cells with mural cells and reconstitution of the surrounding extracellular matrix (ECM). In an adult, angiogenesis normally takes place only in wound healing, tissues repair, and during the female reproductive cycle and pregnancy. In addition, angiogenesis occurs in pathological conditions such as tumor progression, diabetic blindness, age-related macular degeneration, rheumatoid arthritis, psoriasis, and more than 70 other conditions. The balance between the positive and negative regulatory molecules is thought to regulate angiogenesis. The second vascular system of the body, the lymph vascular system, forms during development coincidentally with the maturation of the blood vessels from embryonic veins, through a process called lymphangiogenesis (reviewed in Saharinen et al., 2004).

Positive regulators of angiogenesis are fairly well characterized. Members of the vascular endothelial growth factor (VEGF) family and their receptors function during formation of the initial embryonic vascular plexus, whereas angiopoietins (Angs) and their receptor Tie-2 are implicated in the subsequent remodeling processes (reviewed in (Ferrara et al., Nat. Med., 9:669-676, 2003; Rossant and Howard, Annu. Rev. Cell Dev. Biol., 18:541-573, 2002). Tie-1, an endothelial specific receptor tyrosine kinase, shares high degree of homology with Tie-2. These receptors contain two immunoglobulin-like loops, three EGF-like domains, and three fibronectin type III repeats in their extracellular domains, and tyrosine kinase domains with a number of phosphorylation and protein interactions sites in their cytoplasmic tails. The expression of the tie gene is restricted to the endothelial cells and to some hematopoietic cell lineages (Korhonen et al., Oncogene, 9:395-403, 1994; Partanen et al., Mol. Cell. Biol., 12:1698-1707, 1992). Upregulation of Tie-1 expression has been observed during wound healing, ovarian follicle maturation and tumor angiogenesis (Kaipainen et al., Cancer Res., 54:6571-6577, 1994; Korhonen et al., Blood, 80:2548-2555,1992). Abnormal expression of Ang-2, Tie-1 and Tie-2 was also detected in menorrhagic endometrium (Blumenthal et al., Fertil. Steril., 78:1294-1300, 2002).

Tie-1 is required during the embryonic development for the integrity and survival of vascular endothelial cells, particularly in the regions undergoing angiogenic growth of capillaries. Targeted disruption of the Tie-1 gene in mice results in embryonic lethality between E13.5 and E18.5, depending on the background strain, because of severe edema, extensive hemorrhage and defective microvessel integrity (Puri et al., EMBO J., 14:5884-5891, 1995; Sato et al., Nature, 376:70-74, 1995). The genetic deletion of Tie-2 results in embryonic lethality at E10.5 due to the cardiac failure, hemorrhage, and defects in vascular remodeling and maturation, resulting from improper recruitment of periendothelial supporting cells (Dumont et al., Genes Dev., 8:1897-1909, 1994; Sato et al., Nature, 376:70-74, 1995). Mice lacking both Tie-1 and Tie-2 receptors also die at about E10.5 with similar defects than Tie-2 null animals (Puri et al., Development, 126:4569-4580, 1999).

Tie-1 is an orphan receptor with no reported ligands, whereas three members of the angiopoietin family (Ang-1, Ang-2 and Ang-3/4) have been identified as ligands for Tie-2. Ang-1 and Ang-2 have been extensively studied over the last years. Ang-1 promotes vascular remodeling, maturation, and stabilization of the vasculature, and the Ang-1 null phenotype is very similar but slightly less severe than Tie-2 null phenotype resulting in embryonic lethality at E12.5 (Suri et al., Cell, 87:1171-1180, 1996). Overexpression of Ang-1 under the keratin-14 (K14) promoter in the skin confirms the role of Ang-1 in endothelial proliferation and survival (Thurston et al., Science, 286:2511-2514,1999). Ang-2 is a natural antagonist for Tie-2 in endothelial cells and it is not absolutely required during embryonic development but is necessary during postnatal vascular remodeling. In addition, deletion of Ang-2 results in defects in the patterning and function of the lymphatic vasculature (Gale et al., Dev. Cell., 3:411-423, 2002). The lymphatic defect can be completely rescued by Ang-1, but not the defects in vascular remodeling suggesting that Ang-2 acts as a Tie-2 agonist in the lymphatic vasculature but as an antagonist in the blood vascular system (Gale et al., Dev. Cell., 3:411-423, 2002). Overexpression of Ang-2 in the blood vessels mimics the phenotype of Tie-2 null animals and leads to embryonic lethality at E9.5-E10.5 (Maisonpierre et al., Science, 277:55-60,1997). Ang-1 binding to Tie-2 induces phosphorylation of the receptor while binding of Ang-2 to Tie-2 is unable to induce phosphorylation of the receptor in endothelial cells (Maisonpierre et al., Science, 277:55-60, 1997). None of the angiopoietins have been reported to directly bind Tie-1.

### SUMMARY OF THE INVENTION

The present invention includes compositions and methods of use thereof for the modulation of female fertility and embryogenesis.

In one aspect, the invention is a soluble Tie-1 receptor extracellular domain composition which is useful to inhibit female fertility and embryogenesis. Tie-1-Ig constructs expressed in mice were observed to stabilize ovarian vasculature, inhibiting its regression.

In humans, Tie-1 comprises a receptor tyrosine kinase protein of about 1138 amino acids (Swiss Prot database accession no. P35590 and U.S. Pat. No. 5,955,291, both incorporated herein by reference). This Tie amino acid sequence comprises a signal peptide (aa 1-24) cleaved to yield a mature protein comprised of amino acids 25-1138. The extracellular domain comprises approximately amino acids 25-759, in which residues 43-105 comprises an Ig-like C2-type 1 domain; residues 83, 161, 503, 596, and 709 are putative N-linked glycosylation sites; residues 214-256, 258-303, and 305-345 comprise EGF-like sequences; residues 372-426 comprise an Ig-like C2-type 2 domain; and residues 446-537, 545-637 and 644-736 comprise Fibronectin type-III-like domains. Residues 760-784 comprise the putative transmembrane domain. For the practice of the present invention, fragments of the Tie 1 extracellular domain that are effective for inhibiting fertility or embryogenesis also may be used. Effective fragments may be identified by in vivo screening as described herein. Without being limited to a particular theory, fragments that contain sequences effective to interact with Tie-2 and/or angiopoietin ligands (that bind Tie-1, or Tie-2, or Tie-1/Tie-2 complexes) are specifically contemplated.

In one embodiment, the Tie-1 extracellular domain is fused to an immunoglobulin constant domain (Fc), and preferably to an IgG Fc domain. Fusion to such polypeptides to increase serum half-life (i.e., to slow clearance), is specifically contemplated. Further modifications, including pegylation or addition of other moieties to increase serum half-life also is contemplated.

Variants of the exact human Tie-1 sequence described herein also are contemplated. For example, polypeptides having at least 80%, 85%, 90%, 95%, 96%, 97%, 98%, 99%, or greater percent identity to the Tie-1 receptor extracellular domain sequence described herein, or effective fragments thereof, are specifically contemplated.

The composition preferably further includes a pharmaceutically acceptable diluent, excipient, or carrier.

In a related embodiment, the invention is a soluble Tie-2 receptor extracellular domain composition which is useful to inhibit female fertility and embryogenesis. Human Tie-2 (Swiss Prot database accession no. Q02763, incorporated herein by reference), which has a similar structural organization as Tie-1, comprises an amino acid sequence of 1124 amino acids, of which about residues 1-22 comprise a signal peptide and residues 746-770 comprise the putative transmembrane domain.

For the practice of the present invention, fragments of the Tie-2 extracellular domain that are effective for inhibiting fertility or embryogenesis also may be used. Effective fragments may be identified by in vivo screening (as described herein with respect to Tie-1/Ig peptides). Without being limited to a particular theory, fragments that contain sequences effective to interact with Tie-1 and/or angiopoietin ligands (that bind Tie-2 or Tie-1/Tie-2 complexes) are specifically contemplated.

In one embodiment, the Tie-2 extracellular domain is fused to an immunoglobulin constant domain (Fc), and preferably to an IgG Fc domain. Fusion to such polypeptides to increase serum half-life (i.e., to slow clearance), is specifically contemplated. Further modifications, including pegylation or addition of other moieties to increase serum half life also is contemplated.

Variants of the exact human Tie-2 sequence described herein also are contemplated. For example, polypeptides having at least 80%, 85%, 90%, 95%, 96%, 97%, 98%, 99%, or greater percent identity to the Tie-2 receptor extracellular domain sequence described herein, or effective fragments thereof, are specifically contemplated.

In another embodiment, the invention is the use of Tie-1 or Tie-2 compositions as described here for the manufacture of a medicament to modulate female fertility, e.g., as a contraceptive.

For these and other embodiments where polypeptides are contemplated as therapeutic agent, the invention also includes polynucleotides and vectors (e.g., gene therapy vectors such as adenoviruses, adeno-associated viruses, or lentiviruses) that encode the polypeptides and that can be used to express the polypeptides ex vivo or in vivo. Compositions comprising such polynucleotides or vectors and pharmaceutically acceptable diluents or carriers are contemplated as additional aspects of the invention.

The invention also is a method of inhibiting fertility of a female mammal by administering to the mammal an amount of the polypeptide or polynucleotide materials described herein effective to inhibit fertility. All routes of administration (oral, intravenous intramuscular or other injection, skin patch, topical, vaginal, etc.) are contemplated.

Without intending to be limited to a particular theory, the soluble Tie materials are effective for inhibiting fertility by binding circulating angiopoietin molecules and preventing them from stimulating Tie-1/Tie-2 expressed in the female reproductive system. In another variation, the invention is the use of angiopoietin antibodies or short interfering RNA or antisense molecules or other angiopoietin inhibitors to inhibit female fertility.

The invention also includes compositions comprising an angiopoetin-1 polypeptide for use in manufacture of a medicament to promote fertility and embryogenesis in a subject. The invention further includes compositions comprising an angiopoeitin-2 molecule for use in manufacturing a medicament to promote fertility and embryogenesis in a female subject. In an additional embodiment, the compositions contemplated by the invention further comprise a pharmaceutically acceptable diluent or carrier. The invention includes methods of administering such compositions to a female subject to increase fertility or reduce the likelihood of miscarriages. Administration after ovulation (which can be estimated from body temperature or other monitoring of the female cycle) is specifically contemplated.

As described above with reference to the Tie peptides, the use of fragments and sequence variants for the angiopoietins to treat infertility is specifically contemplated.

Administration of polynucleotides (or vectors) that encode the angiopoietin polypeptides also is contemplated, and use of such polypeptides and polypeptides for manufacture of a medicament to treat infertility is contemplated.

In another aspect, the invention provides a method for modulating female fertility comprising the step of administering to a subject a Tie-1 extracellular domain composition in an amount effective to modulate fertility in the subject. In one aspect, the Tie-1 composition inhibits fertility and inhibits embryogenesis in the subject.

The invention also provides a method for promoting fertility in an subject comprising the step of administering to a subject an Angiopoetin-1 composition in an amount effective to promote fertility in a subject. Promoting fertility includes promoting implantation of an embryo, or promoting growth of an embryo.

Yet another aspect of the invention is a method of screening for infertility in a female, or screening for a biochemical pathway that may be contributing to infertility in a female, comprising measuring Tie receptor expression or activity in a biological sample (e.g., a tissue or fluid sample or biopsy) from a mammalian female, wherein Tie expression or activity correlates with fertility. Teilman and Christensen recently reported in Cell Biol. International (2005) that the Tie-1 and Tie-2 receptors localize to the primary cilia in the female reproductive organs, such as ovarian surface epithelium in humans. Without intending to be limited to a particular theory, aberrant Tie receptor expression or function in these tissues is suggested as causative or correlative with human infertility. In a preferred variation, screening methods are performed using a biological sample that comprises female reproductive tissue, such as ovary, fallopian tube, uterine tissue, or the like. In a highly preferred variation, the biological sample comprises primary cilia of ovarian surface endothelium. In a related variation, the invention comprises analyzing Tie receptor sequence for a mutation that disrupts Tie-1/Tie-2 interactions or Tie/angiopietin interactions.

Yet another variation of the invention is methods of screening for agents that modify female fertility by modulating the interactions between Tie-1 and/or Tie-2 and/or angiopoeitins. More specifically, agents that disrupt the normal interactions between circulating agonist angiopoietin Tie ligands and Tie receptors expressed in the female reproductive system are expected to inhibit fertility and have utility as a contraceptive agent, and agents that mimic or enhance such interactions have utility for promoting fertility.

The following numbered paragraphs summarize additional aspects and embodiments of the invention:

1. A method of modulating fertility or embryogenesis in a mammalian female, comprising:

administering to a mammalian female a medicament comprising a modulator of angiopoietin-induced Tie receptor activity in cells of the female, in an amount effective to modulate fertility or embryogenesis in the female. For the purposes of the invention, “fertility” refers to the ability to conceive and bear viable offspring. The invention is applicable to any mammals but is of particular interest to humans, pets (e.g., dogs, cats), animals of importance to agricultural or sporting (horses, cows, pigs, oxen), endanagered species, and zoo animals. The terms “modulate” refers to both up-regulation (increase fertility) and down-regulation or inhibition (decrease or eliminate fertility).

2. Use of a modulator of angiopoietin-induced Tie receptor activity in the manufacture of a medicament to modulate fertility or embryogenesis in a mammalian female.

3. The method or use of paragraphs 1 or 2, wherein the female is human.

4. The method or use of any one of paragraphs 1-3, wherein the medicament further comprises a pharmaceutically acceptable diluent, excipient or carrier. Appropriate carriers will be apparent for various agents and chosen routes of administration.

5. The method or use of any one of paragraphs 1-4, wherein the modulator is an inhibitor of angiopoietin-induced Tie receptor activity, and the modulator is present in the medicament in an amount effective to inhibit fertility or embryogenesis. Tie receptor activity can be measured in vitro by screening for phosphorylation of the receptor or downstream physiological processes of cells that express the receptor.

6. The method or use of paragraph 5, wherein the inhibitor comprises a soluble polypeptide that binds to an angiopoietin protein and comprises an amino acid sequence that is at least 80% identical to the extracellular domain amino acid sequence of a mammalian Tie-1 or Tie-2 receptor tyrosine kinase.

7. The method or use of paragraph 5, wherein the inhibitor comprises a member selected from the group consisting of:

(A) a polypeptide that comprises:
(i) an amino acid sequence that is at least 80% identical to amino acids 25-759 of SEQ ID NO: 2;
(ii) an amino acid sequence that is at least 80% identical to amino acids 24-745 of SEQ ID NO: 4; and
(iii) fragments of (i) or (ii);
wherein the polypeptide binds at least one angiopoietin polypeptide selected from the group consisting of Angiopoietin-1 (SEQ ID NO: 6), Angiopoietin-2 (SEQ ID NO: 8), Angiopoietin-3 (SEQ ID NO: 10), and Angiopoietin-4 (SEQ ID NO: 12);

(B) polynucleotides that comprise a nucleotide sequence that encode a polypeptide according to (A); and

(C) vectors that comprise a polynucleotide according to (B).

8. A method or use according to paragraph 6 or 7, wherein the polypeptide further comprises an immunoglobulin Fc fragment.

9. The method or use according to paragraph 8, wherein the immunoglobulin Fc fragment comprises an IgG Fc domain.

10. The method or use according to paragraph 5, wherein the inhibitor comprises an antibody substance that specifically immunoreacts to the extracellular domain of a Tie-1 or Tie-2 receptor tyrosine kinase, wherein the antibody substance comprises: (a) a monoclonal or polyclonal antibody; (b) a fragment of (a) that retains said immunoreactivity; or (c) a polypeptide that comprises an antigen binding fragment of (a) and that retains said immunoreactivity.

11. The method according to paragraph 5, wherein the inhibitor comprises an interfering RNA that inhibits expression of a polypeptide selected from the group consisting of a Tie-1 receptor tyrosine kinase, a Tie-2 receptor tyrosine kinase; Angiopoietin-1, Angiopoietin-2, Angiopoietin-3, and Angiopoietin-4.

12. The method or use according to any one of paragraphs 1-4, wherein the modulator is an agonist of Tie receptor activity, and is present in the medicament in an amount effective to increase fertility or promote embryogenesis in the female.

13. The method or use of paragraph 12, wherein the agonist comprises (a) a polypeptide that comprises an amino acid sequence at least 80% identical to a mammalian angiopoietin polypeptide or fragments thereof that is effective to bind and stimulate a Tie receptor tyrosine kinase; or (b) a polynucleotide that comprises a nucleotide sequence that encodes said polypeptide; or (c) a vector that comprises the polynucleotide.

14. The method or use according to paragraph 13, wherein the angiopoietin polypeptide is selected from group consisting of human angiopoietin-1 (SEQ ID NO: 6), angiopoietin-2 (SEQ ID NO: 8), angiopoietin-3 (SEQ ID NO: 10), and angiopoietin-4 (SEQ ID NO: 12).

15. The method or use according to any one of paragraphs 1-14, wherein the medicament is administered orally, by intravenous injection, by intramuscular injection, or other injection, by transdermal patch, topically or vaginally.

16. The method according to any one of paragraphs 1-14, wherein the medicament is administered after ovulation.

17. A method of screening for infertility in a female, comprising measuring Tie receptor expression or activity in a biological sample from a mammalian female, wherein Tie expression or activity correlates with fertility.

18. The method of paragraph 17, wherein the biological sample comprises primary cilia of ovarian surface endothelium.

19. A method of screening for modulators of binding between a Tie receptor tyrosine kinase and an angiopoietin ligand, comprising:

a) contacting a Tie receptor composition with an angiopoietin ligand in the presence and in the absence of a putative modulator compound;

b) measuring binding between the Tie receptor and the angiopoietin ligand in the presence and absence of the putative modulator compound; and

c) identifying a modulator compound based on a decrease or increase in said binding in the presence of the putative modulator compound, as compared to binding in the absence of the putative modulator compound.

20. A method according to paragraph 19, wherein the Tie receptor composition comprises a cell that expresses Tie-1 receptor on its surface.

21. A method according to paragraph 20, wherein the cell further expresses Tie-2 receptor on its surface.

22. A method according to any one of paragraphs 19-21, further comprising a step of:

(d) making a modulator composition by formulating a modulator identified according to step (c) in a pharmaceutically acceptable carrier.

23. A method according to paragraph 22, further comprising a step of:

(e) administering the modulator composition to a mammal that comprises cells that express Tie receptors, and determining physiological effects of the modulator composition in the mammal.

24. A method according to paragraph 23, comprising assessing fertility in mammal.

25. A method according to any one of paragraphs 19-24, wherein the Tie receptor is selected from the group consisting of a mammalian Tie-1 and a mammalian Tie-2 and mixtures thereof.

26. A method according to paragraph 25, wherein the Tie receptor and the angiopoietin are human.

Additional features and variations of the invention will be apparent to those skilled in the art from the entirety of this application, including the detailed description, and all such features are intended as aspects of the invention. It should be understood, however, that the detailed description and the specific examples, while indicating preferred embodiments of the invention, are given by way of illustration only, because various changes and modifications within the spirit and scope of the invention will become apparent to those skilled in the art from this detailed description.

Moreover, features of the invention described herein can be re-combined into additional embodiments that also are intended as aspects of the invention, irrespective of whether the combination of features is specifically mentioned above as an aspect or embodiment of the invention. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention, even if, for brevity, every individual member has not been specifically mentioned herein. When aspects of the invention that are described herein as being selected from a genus, it should be understood that the selection can include mixtures of two or more members of the genus.

In addition to the foregoing, the invention includes, as an additional aspect, all embodiments of the invention narrower in scope in any way than the variations specifically described herein. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### DETAILED DESCRIPTION

The present invention involves the fields of cell and molecular biology, and many standard techniques relevant to those fields will be relevant to the practice of the present invention. Many such techniques are described in Sambrook et al., Molecular Cloning: A Laboratory Manual, Cold Spring Harbor Laboratory Press, Cold Spring Harbor, N.Y. (1989), and/or Ausubel et al., eds., Current Protocols in Molecular Biology, Green Publishers Inc. and Wiley and Sons, NY (1994-2001), both of which are incorporated by reference in their entirety.

A. Gene Sequences of Interest to the Present Invention.

At least two Tie receptors have been identified, referred to as Tie (Tie-1) and Tie-2. The DNA and deduced amino acid sequences of all known Angiopoietins and Tie receptors of any vertebrate species that have been reported in the literature are hereby incorporated by reference. However, due to their special significance to the invention, the following table is provided for the convenience of the reader:

| | Genbank Accession | |
|---|---|---|
| Molecule | Number | SEQ ID NO: |
| Human Tie-1 | NP_005415 | SEQ ID NO: 1 and 2 |
| Human Tie-2 | Q02763; NP_000450 | SEQ ID NO: 3 and 4 |
| Hu Angiopoietin-1 | NM001146 | SEQ ID NO: 5 and 6 |
| Hu Angiopoietin-2 | NM001147 | SEQ ID NO: 7 and 8 |
| Hu Angiopoietin-3 | AF074332 | SEQ ID NO: 9 and 10 |
| Hu Angiopoietin-4 | AF113708 | SEQ ID NO: 11 and 12 |

The Angiopoietin Family Members

The Angiopoietins are of special interest to the present invention because they have been found to modulate (stimulate or inhibit) Tie-2. The angiopoietin (Ang 1-4) family of molecules were originally identified by cDNA library screening for ligands to the orphan Tie 2 receptor tyrosine kinase. [Davis et al., Cell, 87: 1161 69 (1996)]. Ang 1, the first of the angiopoietin ligands identified, was isolated through secretion trap expression cloning using cell lines which demonstrated binding of secreted factors to Tie 2 Fc molecules. This novel technique isolated a 498 amino acid, 70 kDa glycoprotein. The N terminal region of the protein showed hydrophobic sequences characteristic of secretory signal sequences. Residues 100-280 of Ang 1 resemble a coiled coil structure like that found in myosin, while residues 280-498 show homology to a family of proteins which includes fibrinogen, thus this region is the fibrinogen-like domain. Ang-1 shows a binding affinity to Tie 2 less than 4 nM, and induces phosphorylation and activation of the Tie 2 tyrosine kinase.

The remaining members of the angiopoietin family were isolated using homology searches against the Ang-1 cDNA sequence. Human Ang-2, a 496 amino acid protein (Maisonpierre et al, Science. 277: 55 60 (1997)), shows 85% homology to mouse Ang-2 and 60% homology to the Human Ang-1 protein. Ang-2 possesses an amino-terminal secretory signal sequence also found in Ang-1, and also both the coiled coil and fibrinogen-like domains. Ang-2 also shares 8 of the 9 cysteine residues found throughout the Ang-1 sequence, believed to be important in disulfide bond formation. Analysis of Ang-2 activity on the Tie 2 receptor shows that Ang-2 binds to Tie 2 but does not induce phosphorylation of the receptor, implicating Ang-2 as an antagonist to Ang-1 activation of Tie 2.

Angiopoietin 3 has been isolated by several groups based on sequence similarity to Ang-1 and Ang-2. See, e.g., Kim et al., FEBS Lett. 443: 353 6 (1999); Nishimura et al, FEBS Lett. 448: 254 6 (1999). The groups identified either a 503 or 491 amino acid clone of Ang-3, respectively. Nishimura et al. cloned Ang-3 from a human aorta cDNA library, and identified a 503 amino acid protein having 45.1% identity with human Ang-1 and 44.7% identity to Ang-2. A third group independently identified a 460 amino acid Ang-3 clone, (ANGPTL3) from human liver tissue. Conklin et al., Genomics, 62: 477 82 (1999). All three clones possess the characteristic N terminal secretory signal sequence, coiled coil motif, and fibrinogen like domains of the other angiopoietin family members.

Human Ang-4, identified by Valenzuela, et al (Proc. Natl. Acad. Sci USA. 96:1904 09. 1999), using sequence homology to a mouse genomic library, is a 503 amino acid protein having the leader signal sequence, coiled coil, and fibrinogen like sequences indicative of an angiopoietin family member. Both Ang-3 and Ang-4 show conservation of 8 of the 9 cysteines present in Ang-1. Both Ang-3 and Ang-4 have been reported to show binding to the Tie-2 receptor and not Tie-1. Ang-3 acts as an antagonist, while Ang-4 activates Tie-2 as an agonist.

In addition to the foregoing, the invention involves several other polypeptide factors involved in promoting or inhibiting aspects of the angiogenic process. The following description will therefore be useful in the practice of the invention.

With respect to the angiopoietins or other polypeptides used to practice the invention, it will be understood that native sequences will usually be most preferred, but that modifications can be made to most protein sequences without destroying the activity of interest of the protein, especially conservative amino acid substitutions. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine).

Moreover, deletion and addition of amino acids is often possible without destroying a desired activity. With respect to the present invention, where binding activity is of particular interest and the ability of molecules to activate or inhibit receptor tyrosine kinases upon binding is of special interest, binding assays and tyrosine phophorylation assays are available to determine whether a particular ligand or ligand variant (a) binds and (b) stimulates or inhibits RTK activity.

Two manners for defining genera of polypeptide variants include percent amino acid identity to a native polypeptide (e.g., 80, 85, 90, 91, 92, 93, 94, 95, 96, 97, 98, or 99% identity preferred), or the ability of encoding-polynucleotides to hybridize to each other under specified conditions. One exemplary set of conditions is as follows: hybridization at 42° C. in 50% formamide, 5×SSC, 20 mM Na.PO4, pH 6.8; and washing in 1×SSC at 55° C. for 30 minutes. Formula for calculating equivalent hybridization conditions and/or selecting other conditions to achieve a desired level of stringency are well known. It is understood in the art that conditions of equivalent stringency can be achieved through variation of temperature and buffer, or salt concentration as described Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (1989), pp. 9.47 to 9.51.

B. Gene Therapy

While much of the application, including the examples, are written in the context of protein-protein interactions and protein administration, it should be clear that genetic manipulations to achieve modulation of protein expression or activity is specifically contemplated. For example, where administration of proteins is contemplated, administration of a gene therapy vector to cause the protein of interest to be produced in vivo also is contemplated. Where inhibition of proteins is contemplated (e.g., though use of antibodies or small molecule inhibitors), inhibition of protein expression in vivo by genetic techniques, such as knock-out techniques or interfering RNA or anti-sense therapy, is contemplated.

Any suitable vector may be used to introduce a transgene of interest into an animal. Exemplary vectors that have been described in the literature include replication-deficient retroviral vectors, including but not limited to lentivirus vectors [Kim et al., J. Virol., 72(1): 811-816 (1998); Kingsman & Johnson, Scrip Magazine, October, 1998, pp. 43-46.]; adeno-associated viral vectors [Gnatenko et al., J. Investig. Med., 45: 87-98 (1997)]; adenoviral vectors [See, e.g., U.S. Pat. No. 5,792,453; Quantin et al., Proc. Natl. Acad. Sci. USA, 89: 2581-2584 (1992); Stratford-Perricadet et al., J. Clin. Invest., 90: 626-630 (1992); and Rosenfeld et al., Cell, 68: 143-155 (1992)]; Lipofectin-mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in the entirety. Replication-deficient adenoviral vectors and adeno-associated viral vectors constitute preferred embodiments.

In embodiments employing a viral vector, preferred polynucleotides include a suitable promoter and polyadenylation sequence to promote expression in the target tissue of interest. For many applications of the present invention, the Tie promoter (U.S. Pat. No. 5,877,020, incorporated by reference) will be especially suitable. Other suitable promoters/enhancers for mammalian cell expression include, e.g., cytomegalovirus promoter/enhancer [Lehner et al., J. Clin. Microbiol., 29:2494-2502 (1991); Boshart et al., Cell, 41:521-530 (1985)]; Rous sarcoma virus promoter [Davis et al., Hum. Gene Ther., 4:151 (1993)]; or simian virus 40 promoter.

Anti-sense polynucleotides are polynucleotides which recognize and hybridize to polynucleotides encoding a protein of interest and can therefore inhibit transcription or translation of the protein. Full length and fragment anti sense polynucleotides may be employed. Commercial software is available to optimize antisense sequence selection and also to compare selected sequences to known genomic sequences to help ensure uniqueness/specificity for a chosen gene. Such uniqueness can be further confirmed by hybridization analyses. Antisense nucleic acids (preferably 10 to 20 base pair oligonucleotides) are introduced into cells (e.g., by a viral vector or colloidal dispersion system such as a liposome). The antisense nucleic acid binds to the target nucleotide sequence in the cell and prevents transcription or translation of the target sequence. Phosphorothioate and methylphosphonate antisense oligonucleotides are specifically contemplated for therapeutic use by the invention. The antisense oligonucleotides may be further modified by poly-L-lysine, transferrin polylysine, or cholesterol moieties at their 5′ end.

Genetic control can also be achieved through the design of novel transcription factors for modulating expression of the gene of interest in native cells and animals. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular target sequence of the present invention facilitates the engineering of zinc finger proteins specific for the target sequence using known methods such as a combination of structure-based modeling and screening of phage display libraries [Segal et al., (1999) Proc Natl Acad Sci USA 96:2758-2763; Liu et al., (1997) Proc Natl Acad Sci USA 94:5525-30; Greisman and Pabo (1997) Science 275:657-61; Choo et al., (1997) J Mol Biol 273:525-32]. Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence [Segal et al., (1999) Proc Natl Acad Sci USA 96:2758-2763]. The artificial zinc finger repeats, designed based on target sequences, are fused to activation or repression domains to promote or suppress gene expression [Liu et al., (1997) Proc Natl Acad Sci USA 94:5525-30]. Alternatively, the zinc finger domains can be fused to the TATA box-binding factor (TBP) with varying lengths of linker region between the zinc finger peptide and the TBP to create either transcriptional activators or repressors [Kim et al., (1997) Proc Natl Acad Sci USA 94:3616-3620]. Such proteins, and polynucleotides that encode them, have utility for modulating expression in vivo in both native cells, animals and humans. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods [McColl et al., (1999) Proc Natl Acad Sci USA 96:9521-6; Wu et al., (1995) Proc Natl Acad Sci USA 92:344-348].

Another class of therapeutics for inhibiting expression (and therefore activity) of target genes/pathways described herein is interfering RNA technology, also known as RNA interference (RNAi) or short interfering RNA (siRNA).

Using the knowledge of the sequence of target genes such as Tie-1, Tie-2 and Ang-1, siRNA molecules are formed that interfere with the expression of the genes. SiRNA describes a technique by which post-transcriptional gene silencing (PTGS) is induced by the direct introduction of double stranded RNA (dsRNA: a mixture of both sense and antisense strands). (Fire et al., Nature 391:806-811, 1998). Current models of PTGS indicate that short stretches of interfering dsRNAs (21-23 nucleotides; siRNA also known as “guide RNAs”) mediate PTGS. siRNAs are apparently produced by cleavage of dsRNA introduced directly or via a transgene or virus. These siRNAs may be amplified by an RNA-dependent RNA polymerase (RdRP) and are incorporated into the RNA-induced silencing complex (RISC), guiding the complex to the homologous endogenous mRNA, where the complex cleaves the transcript. It is contemplated that RNAi may be used to disrupt the expression of a gene in a tissue-specific manner. By placing a gene fragment encoding the desired dsRNA behind an inducible or tissue-specific promoter, it should be possible to inactivate genes at a particular location within an organism or during a particular stage of development.

In one aspect, the invention provides double-stranded RNA (dsRNA) wherein one strand is complementary to a target region in a target Ang-1, Tie-1 or Tie-2 encoding polynucleotide. In general, dsRNA molecules of this type less than 30 nucleotides in length are referred to in the art as short interfering RNA (siRNA). The invention also contemplates, however, use of dsRNA molecules longer than 30 nucleotides in length, and in certain aspects of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target Ang-1, Tie-1 or Tie-2 encoding polynucleotide. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. Preparation and use of RNAi compounds is described in U.S. Patent Application No. 20040023390, the disclosure of which is incorporated herein by reference in its entirety.

The invention further contemplates methods wherein inhibition of Ang-1, Tie-1 or Tie-2 is effected using RNA lasso technology. Circular RNA lasso inhibitors are highly structured molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target PDGF-B or PDGFR-β-encoding polynucleotide. Because RNA lassos are circular and form tight topological linkage with the target region, inhibitors of this type are generally not displaced by helicase action unlike typical antisense oligonucleotides, and therefore can be utilized as dosages lower than typical antisense oligonucleotides. Preparation and use of RNA lassos is described in U.S. Pat. No. 6,369,038, the disclosure of which is incorporated herein by reference in its entirety.

Anti-sense RNA and DNA molecules, ribozymes, RNAi and triple helix molecules directed against Ang-1, Tie-1 or Tie-2 can be prepared by any method known in the art for the synthesis of DNA and RNA molecules. These include techniques for chemically synthesizing oligodeoxyribonucleotides well known in the art including, but not limited to, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably or transiently into cells.

C. Aptamer Therapeutics

Aptamers are another nucleic acid based method for interfering with Tie/Ang interaction is the use of an aptamer. Aptamers are DNA or RNA molecules that have been selected from random pools based on their ability to bind other molecules. Aptamers have been selected which bind nucleic acid, proteins, small organic compounds, and even entire organisms. Methods and compositions for identifying and making aptamers are known to those of skill in the art and are described e.g., in U.S. Pat. No. 5,840,867 and U.S. Pat. No. 5,582,981 each incorporated herein by reference. Aptamers that bind Tie or Ang are known to those of skill in the art and are specifically contemplated to be useful in the present therapeutic embodiments.

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compared to linear peptide phage display results. Thus, sufficient evidence has been generated to suggest that high affinity ligands can be created and identified by combinatorial molecular evolution techniques. For the present invention, molecular evolution techniques can be used to isolate binding constructs specific for ligands described herein. For more on aptamers, See generally, Gold, L., Singer, B., He, Y. Y., Brody. E., “Aptamers As Therapeutic And Diagnostic Agents,” J. Biotechnol. 74:5-13 (2000). Relevant techniques for generating aptamers may be found in U.S. Pat. No. 6,699,843, which is incorporated by reference in its entirety.

In some embodiments, the aptamer may be generated by preparing a library of nucleic acids; contacting the library of nucleic acids with a growth factor, wherein nucleic acids having greater binding affinity for the growth factor (relative to other library nucleic acids) are selected and amplified to yield a mixture of nucleic acids enriched for nucleic acids with relatively higher affinity and specificity for binding to the growth factor. The processes may be repeated, and the selected nucleic acids mutated and re-screened, whereby a growth factor aptamer is be identified.

D. Antibodies

Antibodies are useful for modulating Tie/Ang interactions due to the ability to easily generate antibodies with relative specificity, and due to the continued improvements in technologies for adopting antibodies to human therapy. Thus, the invention contemplates use of antibodies (e.g., monoclonal and polyclonal antibodies, single chain antibodies, chimeric antibodies, bifunctional/bispecific antibodies, humanized antibodies, human antibodies, and complementary determining region (CDR) grafted antibodies, including compounds which include CDR sequences which specifically recognize a polypeptide of the invention) specific for polypeptides of interest to the invention, especially Tie receptors and angiopoietins. Preferred antibodies are human antibodies which are produced and identified according to methods described in WO93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind the polypeptide of interest preferentially and substantially exclusively (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds), Antibodies A Laboratory Manual; Cold Spring Harbor Laboratory; Cold Spring Harbor, N.Y. (1988), Chapter 6. Antibodies of the invention can be produced using any method well known and routinely practiced in the art.

A monoclonal antibody to a Tie or angiopoietin protein may be prepared by using any technique which provides for the production of antibody molecules by continuous cell lines in culture. These include but are not limited to the hybridoma technique originally described by Köhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred. “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. (Nature 321: 522-525, 1986), Riechmann et al., (Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149 165, 1994. Further changes can then be introduced into the antibody framework to modulate affinity or immunogenicity.

E. Dosing

Polypeptides according to the invention may be administered in any suitable manner using an appropriate pharmaceutically-acceptable vehicle, e.g., a pharmaceutically-acceptable diluent, adjuvant, excipient or carrier. The composition to be administered according to methods of the invention preferably comprises (in addition to the polynucleotide or vector) a pharmaceutically-acceptable carrier solution such as water, saline, phosphate-buffered saline, glucose, or other carriers conventionally used to deliver therapeutics.

The “administering” that is performed according to the present invention may be performed using any medically-accepted means for introducing a therapeutic directly or indirectly into a mammalian subject, including but not limited to injections (e.g., intravenous, intramuscular, subcutaneous, or catheter); vaginal administration; oral ingestion; intranasal or topical administration; and the like. The therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly, although administration following ovulation is preferred.

Polypeptides for administration may be formulated with uptake or absorption enhancers to increase their efficacy. Such enhancer include for example, salicylate, glycocholate/linoleate, glycholate, aprotinin, bacitracin, SDS caprate and the like. See, e.g., Fix (J. Pharm. Sci., 85(12) 1282-1285, 1996) and Oliyai and Stella (Ann. Rev. Pharmacol. Toxicol., 32:521-544, 1993).

The amounts of peptides in a given dosage will vary according to the size of the individual to whom the therapy is being administered as well as the serum half life and potency of the agent. A medicament may be administered as a single dosage form or as multiple doses. Standard dose-response studies, first in animal models such as mice or rats and then primates and then in clinical testing, reveal optimal dosages.

F. Kits

As an additional aspect, the invention includes kits which comprise compounds or compositions of the invention packaged in a manner which facilitates their use to practice methods of the invention. In a simplest embodiment, such a kit includes a compound or composition described herein as useful for practice of a method of the invention (e.g., polynucleotides or polypeptides for administration to a person), packaged in a container such as a sealed bottle or vessel, with a label affixed to the container or included in the package that describes use of the compound or composition to practice the method of the invention. Preferably, the compound or composition is packaged in a unit dosage form. The kit may further include a device suitable for administering the composition according to a preferred route of administration.

Compounds of compositions of the invention also may be packaged with or in admixture with other materials and methods for modulating female fertility, such as natural or synthetic hormones, including but not limited to ethinyl estradiol (EE), estrane progestins, levnorgestrels, and the like.

Additional aspects and details of the invention will be apparent from the following examples, which are intended to be illustrative rather than limiting.

### EXAMPLE 1

In order to clarify the function of Tie-1 a mouse line was generated, which expresses an extracellular domain of human Tie-1 (tyrosine kinase with Ig and EGF homology domains 1) receptor fused to the human IgG Fc region under the K14 promoter in dermal keratinocytes. Expression of this construct in vivo is expected to result in the secretion of the soluble receptor molecule into the dermis and diffusion eventually into the blood stream and various tissue fluids where it would be able to trap possible ligand molecules and prevent their interaction with the endogenous receptor. Three different founder lines were used. The K14-Tie-1/Fc mice in FVB/N background were viable and appeared normal. However, while breeding this transgenic mouse line it became evident that the females were unable to produce progeny and the transgene was transferred to the next generation only via the males. Transgenic females from two different founder lines were mated with a transgenic male seven times. Each time, a plug was observed, but in only one of the females two embryos were found at E18.5, while no progeny was produced in the six matings. In contrast, when a transgenic male was mated with a FVB/N female, each of the fifteen mating resulted with a normal size litter (between 6 and 12 pups/litter, female:male ratio about 50:50).

To define the problem leading to infertility of the females, implantation of the embryo was studied. To this end, both transgenic and normal FVB/N females were super-ovulated and mated with normal FVB/N males. At E7.5 the animals were sacrificed and utero were removed for histological analysis. Embryos had implanted and appeared normal in both transgenic and non-transgenic utero, indicating that implantation takes places normally in these mice. However, no signs of the embryos were observed at E12.5.

When analyzing the ovaries after the super-ovulation, an abnormal luteinization in the transgenic animals was observed, which was not seen in the normal FVBN females. In addition, cyst formation was detected in the ovaries. Furthermore, the uterus had cyst formation surrounded by thin endometrium.

The expression of the soluble Tie-1 receptor under the K14 promoter in the skin of transgenic mice resulted in infertility of the females. The mice appeared otherwise normal, and the males were fertile and able to transfer the transgene to the next generation. Also, the same transgenic males, when mated with transgenic females and producing no progeny, were able to produce normal progeny with normal FVB/N females indicating problems with the female mice. The ovaries showed massive luteinization with some maturing follicles of fairly normal appearance. However, the number of follicles seemed to be somewhat decreased compared to the wild type ovaries. It seems that the implantation of the embryos occurred subnormally; there were fewer implanted embryos in the transgenic utero than in the normal utero. No embryos were detected at E12.5, indicating problems in the post-implantation events. These observations also suggest that the sperm was not defective. Because the transgene expression in the embryos starts between E14 and E15, i.e., after the abortion of the transgenic progeny, and because not only the transgenic embryos get aborted, these results indicate that the infertility is due to the transgene expression in the mother.

Tie-1 and Tie-2 have been shown to form heterodimers as described below in Example 2 and in (Marron et al., 2000). No ligand has been reported for Tie-1, and none of the Tie-2 ligands are reported to bind directly to Tie-1, although, curiously, Tie-1 is phosphorylated upon Ang-1 or Ang-4 stimulation, as described below in Example 2. However, Ang-2 expression is readily detectable only in ovary, placenta, and uterus, which are the predominant sites of vascular remodeling in the normal adult, and the site where we see a phenotype in K14-Tie-1/Fc animals. Furthermore, Ang-2 mRNA expression is highly upregulated in the aged corpus luteum in which blood vessels degenerate. It is plausible that even if there is no direct binding of the angiopoietins to Tie-1, there exist a Tie-1/Tie-2 complex, which generates specific signals in the presence of Ang-2 and/or Ang-1. We are proposing a model in which the overexpression of the soluble Tie-1 receptor in the transgenic animals results in the abolishment of the signaling through endogenous Tie-1 receptor leading to sustained corpus luteum in the ovaries. The massive luteinization of the ovaries supports this idea and that probably leads to improper hormone production by the ovaries. The phenotype is very similar to that obtained in a transgenic mouse overexpressing the human chorionic gonadotropin, which also causes infertility of the females (Rulli et al., 2002). Furthermore, the placentation of the embryos could be defective in these transgenic animals.

Administration of a soluble Tie-1 extracellular domain construct (or the in vivo expression of same via gene therapy) in wildtype female adult mice can be preformed to rule out the possibility that the presence of the soluble Tie-1 receptor would lead to defective development of the ovaries/uterus in the transgenic mice.

Results with the K14-Tie1/Fc transgenic mice indicate that blocking the signaling through Tie-1 receptor caused infertility in females, which indicates that soluble Tie1 has an indication as a contraceptive agent. The molecular mechanisms underlying this phenomenon also will be used to enhance fertility.

### EXAMPLE 2

### Tie-1 Interactions with Tie-2 and Angiopoietins

Experiments were conducted to evaluate and characterize Tie-1 interactions with Tie-2 and with angiopoietin family members. The results, summarized herein, are described in greater detail in Saharinen et al., 2005, J. Cell Biol., 169(2): 239-43, incorporated herein by reference in its entirety.

Materials and Methods

293, 293T (American Type Culture Collection), and EA.hy926 immortalized hybrid HUVECs (Edgell et al., 1983) were grown in DME supplemented with 10% FBS (PromoCell). HUVECs were cultured as described in (Marron et al., 2000, J. Biol. Chem., 275: 39741-39746). LEC, BEC (Makinen et al., 2001, EMBO J., 20: 4762-4773), and HMEC-1 human dermal microvascular cells immortalized with SV40 Large T antigen (Ades et al., 1992, J. Invest. Dermatol., 99: 683-690) were grown in Endothelial Cell Basal Medium (PromoCell) with supplements provided by the manufacturer. Confluent plates of cells were serum-starved overnight, followed by ligand stimulation for 15 minutes, unless otherwise indicated.

The following reagents were used: Tie-1-Fc, Tie-2-Fc, Ang-1, VEGF (all from R&D Systems), Ang-2, Ang-3, Ang-4 (Lee et al., FASEB J., 18: 1200-1208.2004), COMP-HFARP (Kim et al., 2000, Biochem. J., 346:603-610), and Ang-2 (Scharpfenecker et al., 2005, J. Cell Sci., 188:771-780).

The following antibodies were used: antiphosphotyrosine (4G10; Upstate Biotechnology), anti-Tie-1 and anti-Tie-2 (R&D Systems; Santa Cruz Biotechnology, Inc.; clone 33 [Upstate Biotechnology]), anti-V5 (Invitrogen), and anti-Tie-2 (Harris et al., 2001, Clin. Cancer Res., 7: 1992-1997).

Cells were transfected using Fugene6 (Roche Diagnostics), changed to serum-free medium after 48 hours, and harvested 72 hours after transfection. Kinase-inactivating mutation in human Tie-2 (lysine 855 to arginine), human Tie-1 (lysine 870 to arginine), Tie1-V5, and Tie2-Myc constructs were created by PCR. All constructs were confirmed by sequencing (Applied Biosystems).

For immunoprecipitation and immunoblotting, cells were lysed in lysis buffer (50 mM Hepes, pH 7.5, 1% Triton X-100, 5% glycerol, 1 mM EGTA, 150 mM NaCl, 1.5 mM MgCl2, 100 mM NaF, 1 mM Na3VO4, PMSF, aprotinin, and leupeptin) or alternatively in SDS-lysis buffer (Saharinen et al., 1997, Blood, 90: 4341-4353). Equal amounts of cell lysate protein were pre-cleared by incubation with protein G-Sepharose (Amersham Biosciences), followed by addition of BSA (1%) and specific antibodies. The immunocomplexes, captured by protein G-Sepharose, were separated in 7.5% SDS-PAGE (Ready-Gels; Bio-Rad Laboratories) and blotted and detected using specific primary antibodies, biotinylated anti-mouse or anti-goat secondary antibodies (DakoCytomation), and streptavidinbiotin HRP conjugate (Amersham Biosciences) followed by ECL detection with the SuperSignal West Femto Maximun Sensitivity Substrate (Pierce Chemical Co.).

HUVECs were cross-linked in PBS containing 0.5 mM DTSSP for 30 minutes, quenched by addition of Tris, pH 7.5, to 100 mM, and lysed in 50 mM Tris, pH 7.4, 50 mM NaCl, 1% Triton X-100, 1 mM sodium orthovanadate, 1 mM sodium fluoride, 1 mM EGTA, and complete protease inhibitor.

293T cells were cross-linked for 40 min with 1 mM DTSSP on ice.

For RNA isolation and Northern blotting, total RNA was isolated using the RNeasy kit (QIAGEN), electrophoresed, blotted, and hybridized with 32P-labeled cDNA probes.

Results

To investigate the signal transduction pathways of Tie-1, human dermal blood vascular endothelial cells (BEC) and lymphatic endothelial cells (LEC; Makinen et al., 2001, EMBO J., 20: 4762-4773) were stimulated with a COMP-Ang-1 chimeric protein (Cho et al., 2004, Proc. Natl. Acad. Sci. USA., 101: 5547-5552; Cho et al., 2004, Proc. Natl. Acad. Sci. USA., 101: 5553-5558, both incorportated herein by reference).

Surprisingly, COMP-Ang-1 induced tyrosine phosphorylation of Tie-1, in addition to phosphorylation of Tie-2. Phosphorylation of Tie-1 occurred in endothelial cells within 5 minutes of COMP-Ang-1 stimulation, reaching a maximum level at 1 hour, followed by a gradual down-regulation. The kinetics of Tie-2 phosphorylation paralleled these changes observed for Tie-1. Significant phosphorylation occurred with a 100 ng/ml concentration of COMP-Ang-1, but maximal phosphorylation of both receptors required 600 ng/ml. COMP-Ang-1 also induced phosphorylation of Tie-1 and Tie-2 in the hybrid endothelial cell line EA.hy926.

In contrast, 600 ng/ml Ang-2 did not activate either Tie-1 or Tie-2. In fact, decreased Tie-1 phosphorylation was seen when COMP-Ang-1 was provided in combination with an excess of Ang-2.

The soluble extracellular domain of Tie-2 (Tie-2-Fc) has been found to bind Ang-1 and to inhibit Ang-1-induced Tie-2 activation, whereas no effect has been found with the soluble Tie-1 receptor (Davis et al., 1996; Peters et al., 2004). Tie-2-Fc inhibited COMP-Ang-1-induced Tie-1 and Tie-2 phosphorylation, whereas Tie-1-Fc had little if any effect, indicating that COMP-Ang-1 binds to the soluble form of Tie-2 but not to soluble Tie-1, although COMP-Ang-1 was capable of inducing activation of Tie-1 at the cell surface.

To understand the mechanism of COMP-Ang-1-induced Tie-1 activation, Tie-1 was over-expressed in 293T cells, which lack both Tie-1 and Tie-2. Variable and low levels of Tie-1 tyrosine phosphorylation were detected after stimulation of these cells with 600 ng/ml of COMP-Ang-1. This finding suggested that over-expressed Tie-1 can be activated to some degree by high concentrations of COMP-Ang-1 in the absence of Tie-2.

The effect of Tie-2 on COMP-Ang-1 activation of Tie-1 in the transfected cells was examined. Because of the strong basal autophosphorylation of Tie-2 observed in 293T cells, 293 cells that do not replicate transiently transfected expression plasmids were used. The 293 cells were transfected with vectors encoding Tie-1, Tie-2, or both receptors, and stimulated with COMP-Ang-1. COMP-Ang-1-induced tyrosine phosphorylation of Tie-1 was increased in the double transfected cells in comparison with cells transfected only with Tie-1, suggesting that heteromerization of Tie-1 and Tie-2 enhances Tie-1 activation. In contrast, Tie-2 phosphorylation was not enhanced by the presence of Tie-1 when compared with cells transfected with Tie-2 alone.

It was possible that Tie-2 was required for high-affinity binding of COMP-Ang-1 to Tie-1, or that Tie-2 induced the phosphorylation and thereby enhanced the activation of Tie-1 in a Tie-1-Tie-2 complex. To analyze this hypothesis, K870R-Tie-1 was expressed with or without Tie-2. This Tie-1 variant has an inactivating substitution in the kinase domain. K870R-Tie-1 was phosphorylated in a ligand-dependent manner when coexpressed with Tie-2, whereas no phosphorylation was detected in the absence of Tie-2. Thus, Tie-2 was able to induce Tie-1 phosphorylation.

A kinase-inactive K855R-Tie-2 was tested to determine if it, like wild-type Tie-2, was able to enhance Tie-1 phosphorylation. Tie-1 phosphorylation was reduced when it was co-expressed with K855R-Tie-2, indicating that the kinase activity of Tie-2 is required for full enhancement of Tie-1 activation by COMP-Ang-1.

The results obtained from the transfected cells suggested that Tie-1 and Tie-2 undergo heteromerization when stimulated by COMP-Ang-1. To analyze this finding, 293T cells transfected with Tie-1-V5 and Tie-2-Myc constructs were used. After COMP-Ang-1 stimulation, the cell surface proteins were chemically cross-linked with 3,3′-dithiobis[sulfosuccinimidylpropionate] (DTSSP), a membrane non-permeable cross-linker, and Tie-1 was immunoprecipitated from the cell lysates. Interestingly, Tie-2 was co-precipitated with Tie-1 from the double transfected cells. The treatment of human umbilical vein endothelial cells (HUVECs) with DTSSP resulted in co-precipitation of Tie-1 with Tie-2, whereas no co-precipitation was found in non-treated cells. This evidence indicates that Tie-1 and Tie-2 form heteromeric complexes on the cell surface.

These results also suggest that, in the heteromeric complexes, Tie-2 directly phosphorylates Tie-1, as Tie-2 induced phosphorylation of kinase-inactive Tie-1 in a COMPAng-1-dependent manner. COMP-Ang-1 has been shown to be a more potent angiopoietin ligand than native Ang-1 (Cho et al., 2004).

Experiments also were conducted to analyze whether native Ang-1 can induce Tie-1 phosphorylation. Native Ang-1 induced Tie-1 phosphorylation in endothelial cells, although several-fold less efficiently than COMP-Ang-1. The chimeric protein COMP-HFARP (hepatic fibrinogen/angiopoietin-related protein) that does not bind to Tie-1 or Tie-2 (Kim et al., 2000) had no effect even at high concentrations. Thus, COMP-Ang-1-induced Tie-1 activation is mediated via Ang-1 and not by the COMP domain. In addition to Ang-1, Ang-4 is a ligand for human Tie-2, whereas Ang-3 is a specific ligand for murine Tie-2 (Lee et al., 2004, FASEB J., 18:1200-1208.). In additional experiments, Tie-1 phosphorylation was induced by native Ang-4, but not by Ang-3 or Ang-2.

### REFERENCES

Blumenthal, R. D., Taylor, A. P., Goldman, L., Brown, G., and Goldenberg, D. M. (2002). Abnormal expression of the angiopoietins and Tie receptors in menorrhagic endometrium. Fertil Steril 78, 1294-1300.
Dumont, D. J., Gradwohl, G., Fong, G. H., Puri, M. C., Gertsenstein, M., Auerbach, A., and Breitman, M. L. (1994). Dominant-negative and targeted null mutations in the endothelial receptor tyrosine kinase, tek, reveal a critical role in vasculogenesis of the embryo. Genes Dev 8, 1897-1909.
Ferrara, N., Gerber, H. P., and LeCouter, J. (2003). The biology of VEGF and its receptors. Nat Med 9, 669-676.
Gale, N. W., Thurston, G., Hackett, S. F., Renard, R., Wang, Q., McClain, J., Martin, C., Witte, C., Witte, M. H., Jackson, D., et al. (2002). Angiopoietin-2 is required for postnatal angiogenesis and lymphatic patterning, and only the latter role is rescued by Angiopoietin-1. Dev Cell 3, 411-423.
Kaipainen, A., Vlaykova, T., Hatva, E., Bohling, T., Jekunen, A., Pyrhonen, S., and Alitalo, K. (1994). Enhanced expression of the tie receptor tyrosine kinase mesenger RNA in the vascular endothelium of metastatic melanomas. Cancer Res 54, 6571-6577.
Korhonen, J., Partanen, J., Armstrong, E., Vaahtokari, A., Elenius, K., Jalkanen, M., and Alitalo, K. (1992). Enhanced expression of the tie receptor tyrosine kinase in endothelial cells during neovascularization. Blood 80, 2548-2555.
Korhonen, J., Polvi, A., Partanen, J., and Alitalo, K. (1994). The mouse tie receptor tyrosine kinase gene: expression during embryonic angiogenesis. Oncogene 9, 395-403.
Maisonpierre, P. C., Suri, C., Jones, P. F., Bartunkova, S., Wiegand, S. J., Radziejewski, C., Compton, D., McClain, J., Aldrich, T. H., Papadopoulos, N., et al. (1997). Angiopoietin-2, a natural antagonist for Tie2 that disrupts in vivo angiogenesis. Science 277, 55-60.
Marron, M. B., Hughes, D. P., Edge, M. D., Forder, C. L., and Brindle, N. P. (2000). Evidence for heterotypic interaction between the receptor tyrosine kinases TIE-1 and TIE-2. J Biol Chem 275, 39741-39746.
Partanen, J., Armstrong, E., Makela, T. P., Korhonen, J., Sandberg, M., Renkonen, R., Knuutila, S., Huebner, K., and Alitalo, K. (1992). A novel endothelial cell surface receptor tyrosine kinase with extracellular epidermal growth factor homology domains. Mol Cell Biol 12, 1698-1707.
Puri, M. C., Partanen, J., Rossant, J., and Bernstein, A. (1999). Interaction of the TEK and TIE receptor tyrosine kinases during cardiovascular development. Development 126, 4569-4580.
Puri, M. C., Rossant, J., Alitalo, K., Bernstein, A., and Partanen, J. (1995). The receptor tyrosine kinase TIE is required for integrity and survival of vascular endothelial cells. Embo J 14, 5884-5891.
Rossant, J., and Howard, L. (2002). Signaling pathways in vascular development. Annu Rev Cell Dev Biol 18, 541-573.
Rulli, S. B., Kuorelahti, A., Karaer, O., Pelliniemi, L. J., Poutanen, M., and Huhtaniemi, I. (2002). Reproductive disturbances, pituitary lactotrope adenomas, and mammary gland tumors in transgenic female mice producing high levels of human chorionic gonadotropin. Endocrinology 143, 4084-4095.
Sato, T. N., Tozawa, Y., Deutsch, U., Wolburg-Buchholz, K., Fujiwara, Y., Gendron-Maguire, M., Gridley, T., Wolburg, H., Risau, W., and Qin, Y. (1995). Distinct roles of the receptor tyrosine kinases Tie-1 and Tie-2 in blood vessel formation. Nature 376, 70-74.
Suri, C., Jones, P. F., Patan, S., Bartunkova, S., Maisonpierre, P. C., Davis, S., Sato, T. N., and Yancopoulos, G. D. (1996). Requisite role of angiopoietin-1, a ligand for the TIE2 receptor, during embryonic angiogenesis. Cell 87, 1171-1180.
Thurston, G., Suri, C., Smith, K., McClain, J., Sato, T. N., Yancopoulos, G. D., and McDonald, D. M. (1999). Leakage-resistant blood vessels in mice transgenically overexpressing angiopoietin-1. Science 286, 2511-2514.

All documents cited herein are hereby incorporated by reference in their entirety.

The invention has been described with reference to specific embodiments and experiments. However, the foregoing description should be understood to be exemplary and not limiting. The only limitations defining or placed on the invention are those in the claims.

1. A method of modulating fertility or embryogenesis in a mammalian female, comprising:
administering to a mammalian female a composition comprising a modulator of angiopoietin-induced Tie receptor activity in cells of the female, in an amount effective to modulate fertility or embryogenesis in the female.

2. (canceled)

3. The method of claim 1, wherein the female is human.

4. The method of claim 1, wherein the composition further comprises a pharmaceutically acceptable diluent, excipient or carrier.

5. The method of claim 1, wherein the modulator is an inhibitor of angiopoietin-induced Tie receptor activity, and the modulator is present in the composition in an amount effective to inhibit fertility or embryogenesis.

6. The method of claim 5, wherein the inhibitor comprises a soluble polypeptide that binds to an angiopoietin protein and comprises an amino acid sequence that is at least 80% identical to the extracellular domain amino acid sequence of a mammalian Tie-1 or Tie-2 receptor tyrosine kinase.

7. The method of claim 5, wherein the inhibitor comprises a member selected from the group consisting of:
(A) a polypeptide that comprises:
(i) an amino acid sequence that is at least 80% identical to amino acids 25-759 of SEQ ID NO: 2;
(ii) an amino acid sequence that is at least 80% identical to amino acids 23-745 of SEQ ID NO: 4; and
(iii) fragments of (i) or (ii);
wherein the polypeptide binds at least one angiopoietin polypeptide selected from the group consisting of Angiopoietin-1 (SEQ ID NO: 6), Angiopoietin-2 (SEQ ID NO: 8), Angiopoietin-3 (SEQ ID NO: 10), and Angiopoietin-4 (SEQ ID NO: 12);
(B) a polynucleotide comprising a nucleotide sequence that encode a polypeptide according to (A); and
(C) a vector comprising a polynucleotide according to (B).

8. A method according to claim 6, wherein the polypeptide further comprises an immunoglobulin Fc fragment.

9. The method according to claim 8, wherein the immunoglobulin Fc fragment comprises an IgG Fc domain.

10. The method according to claim 5, wherein the inhibitor comprises an antibody substance that specifically immunoreacts to the extracellular domain of a Tie-1 or Tie-2 receptor tyrosine kinase, wherein the antibody substance comprises: (a) a monoclonal or polyclonal antibody; (b) a fragment of (a) that retains said immunoreactivity; or (c) a polypeptide that comprises an antigen binding fragment of (a) and that retains said immunoreactivity.

11. The method according to claim 5, wherein the inhibitor comprises an interfering RNA that inhibits expression of a polypeptide selected from the group consisting of a Tie-1 receptor tyrosine kinase, a Tie-2 receptor tyrosine kinase; Angiopoietin-1, Angiopoietin-2, Angiopoietin-3, and Angiopoietin-4.

12. The method according to claim 1, wherein the modulator is an agonist of Tie receptor activity, and is present in the composition in an amount effective to increase fertility or promote embryogenesis in the female.

13. The method of claim 12, wherein the agonist comprises (a) a polypeptide that comprises an amino acid sequence at least 80% identical to a a mammalian angiopoietin polypeptide or fragments thereof that is effective to bind and stimulate a Tie receptor tyrosine kinase; or (b) a polynucleotide that comprises a nucleotide sequence that encodes said polypeptide; or (c) a vector that comprises the polynucleotide.

14. The method according to claim 13, wherein the angiopoietin polypeptide is selected from group consisting of human angiopoietin-1 (SEQ ID NO: 6), angiopoietin-2 (SEQ ID NO: 8), angiopoietin-3 (SEQ ID NO: 10), and angiopoietin-4 (SEQ ID NO: 12).

15. The method according to claim 1, wherein the medicament is administered orally, by intravenous injection, by intramuscular injection, or other injection, by transdermal patch, topically or vaginally.

16. The method according to claim 1, wherein the medicament is administered after ovulation.

17. A method of screening for infertility in a female, comprising measuring Tie receptor expression or activity in a biological sample from a mammalian female, wherein Tie expression or activity correlates with fertility.

18. The method of claim 17, wherein the biological sample comprises primary cilia of ovarian surface endothelium.

19. A method of screening for modulators of binding between a Tie receptor tyrosine kinase and an angiopoietin ligand, comprising:
a) contacting a Tie receptor composition with an angiopoietin ligand in the presence and in the absence of a putative modulator compound;
b) measuring binding between the Tie receptor and the angiopoietin ligand in the presence and absence of the putative modulator compound; and
c) identifying a modulator compound based on a decrease or increase in said binding in the presence of the putative modulator compound, as compared to binding in the absence of the putative modulator compound.

20. A method according to claim 19, wherein the Tie receptor composition comprises a cell that expresses Tie-1 receptor on its surface.

21. A method according to claim 20, wherein the cell further expresses Tie-2 receptor on its surface.

22. A method according to claim 19, further comprising a step of:
(d) making a modulator composition by formulating a modulator identified according to step (c) in a pharmaceutically acceptable carrier.

23. A method according to claim 22, further comprising a step of:
(e) administering the modulator composition to a mammal that comprises cells that express the Tie receptor, and determining physiological effects of the modulator composition in the mammal.

24. A method according to claim 23, comprising assessing fertility in mammal.

25. A method according to claim 19, wherein the Tie receptor is selected from the group consisting of a mammalian Tie-1, a mammalian Tie-2 and mixtures thereof.

26. A method according to claim 25, wherein the Tie receptor and the angiopoietin are human.
